(19) United States
(12) Patent Application Publication
(10) Pub. No.: US 2026/0256735 A1
(43) Pub. Date: Sep. 3, 2026

(54) TOPICAL PHARMACEUTICAL COMPOSITION COMPRISING AN ASSOCIATION OF AN ISOXAZOLINE WITH A MACROCYCLIC LACTONE AND A PYRAZINO-ISOQUINOLINE DERIVATIVE FOR TREATING PARASITIC INFESTATIONS IN SMALL ANIMALS

(71) Applicant: AGROVET MARKET S.A., San Luis, Lima (PE)

(72) Inventors: Jorge Umberto CALDERON OJEDA, La Molina Vieja Lima (PE); Karen Melissa DELGADO CRISPIN, San Juan de Miraflores Lima (PE); Erick Alfredo MOLLEAPAZA AGUILAR, Segunda etapa Lima (PE)

(73) Assignee: AGROVET MARKET S.A., San Luis, Lima (PE)

(21) Appl. No.: 19/157,646

(22) PCT Filed: Sep. 25, 2024
(86) PCT No.: PCT/PE2024/050019

§ 371 Date: Aug. 18, 2025

(30) Foreign Application Priority Data
Dec. 22, 2023 (PE) 003466-2023/DIN

(51) Int. Cl. A61K 31/365; A61K 31/42; A61K 31/4985; A61P 33/10; A61P 33/14

(52) U.S. Cl. CPC A61K 31/365; A61K 31/42; A61K 31/4985; A61P 33/10; A61P 33/14

(57) ABSTRACT

The present invention relates to a pharmaceutical composition or formulation and to a method for manufacturing or producing same, wherein the pharmaceutical composition comprises a synergic association of an isoxazoline with a macrocylcic lactone and a synthetic pyrazino-isoquinoline derivative, wherein the composition is in the form of a topical solution for localised topical application, called spot-on, which has the feature of high skin permeability when applied to the skin of the animal. The invention also relates to the uses thereof to prevent and/or treat infestation of small animals by external and internal parasites.

### TECHNICAL FIELD

The present invention pertains to the technical field of the pharmaceutical industry, mainly with the industry of pharmaceutical products for veterinary use.

### PRIOR ART

The development of pharmaceutical compositions or formulations comprising an association of antiparasitic agents that have synergistic activity against internal parasitosis caused by flatworms and other species of acinthes worms; as well as against external parasitosis caused by fleas, ticks and mites, is a current need for the treatment and control of infestations in small animals.

Within the scope of patent literature is U.S. Pat. No. 11,464,763 which relates to topical compositions for combating ectoparasites and endoparasites in animals, comprising at least one isoxazoline active agent and a pharmaceutically acceptable vehicle, optionally in combination with one or more additional active agents. This invention also provides methods for eradicating, controlling and preventing infections and infestations by parasites in an animal comprising administering the compositions of the invention to the animal in need thereof.

Also, document U.S. Pat. No. 9,682,949 refers to processes for the preparation of new insecticidally active thiethane derivatives. The invention also relates to thiethane derivatives and to intermediates used in the preparation of thiethane derivatives, as well as to methods for using thiethane derivatives to combat and control insect, mite, nematode, and mollusk pests.

Patent U.S. Pat. No. 8,466,115 provides spirocyclic isoxazoline-derived compounds and their stereoisomers, which act as parasiticides, in particular, ectoparasiticides; therefore, it can be used to prevent, treat, repel, and control mite and insect infections and infestations in animals. In addition, the invention contemplates the control and prevention of tick-borne diseases, for example, Lyme disease, canine and bovine anaplasmosis, canine ehrlichiosis, canine rickettsiosis, canine and bovine babesiosis, bovine epizootic abortion and theileriosis. The compounds can be administered topically through a support matrix individually or with veterinarily acceptable excipients, diluent, or carriers optionally with additional veterinary agents or salts thereof.

Patent EP2619189 discloses isoxazoline oxime derivatives having parasiticidal activity. The compounds of interest are isoxazoline oxymadione derivatives. The invention also relates to compositions and methods of use thereof for treating or preventing a parasitic infection or infestation in an animal including the step of administering to said animal, in need of said treatment, a therapeutically effective amount of a compound derived from isoxazoline, geometric isomer, stereoisomer thereof or pharmaceutically or veterinarily acceptable salt thereof for oral, topical, and subcutaneous administration.

The document MX2013001437 discloses isoxazoline-substituted azetidine derivative, stereoisomers thereof and veterinarily acceptable salts thereof and their use as a parasiticide in mammals and birds.

The document U.S. Pat. No. 9,376,434 discloses dihydroazole compounds of formula (I) that are biologically active against endoparasites and ectoparasites that damage animals and against pests that damage crops and also teaches parasiticidal and pesticidal compositions comprising the dihydroazole compounds in combination with a pharmaceutically acceptable vehicle or an agriculturally acceptable vehicle and a method comprising administering an effective amount of a compound of the invention to the animal or plants, or to the soil in which the infected plant grows.

In this sense, there is still a need to provide a composition or pharmaceutical formulation for topical administration comprising an association of active ingredients that can provide comprehensive protection against external parasites such as fleas, ticks, and mites, as well as internal parasites such as Dirofilariasis, gastrointestinal nematodes, and tapeworms, among others. The proposed composition contains an active of the isoxazoline family, a macrocyclic lactone and a syntheticpyrazino-isoquinolinederivative.

The proposed composition is a broad-spectrum parasiticide containing an association of an isoxazoline with at least one macrocyclic lactone and at least one pyrazino-isoquinoline derivative available for topical application. The composition of topical administration where it has a high permeability at cutaneous level when applied on the skin of the animal, and its uses in the prevention and/or treatment of infestation of domestic animals by external and internal parasites.

### DESCRIPTION OF THE DRAWING

FIG. 1 shows the area counts that were performed at six locations on each animal: neck, dorsal midline, tail base, left lateral, right lateral, and inguinal area.

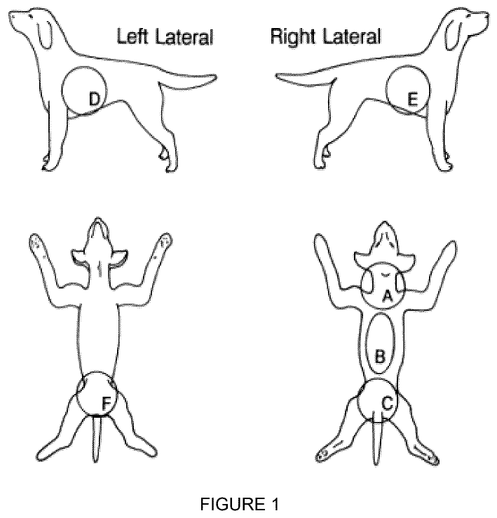

### BRIEF DESCRIPTION OF THE INVENTION

The present invention is directed to a pharmaceutical formulation or composition and a method of manufacture or production thereof, wherein the pharmaceutical composition comprises at least one isoxazoline, at least one macrocyclic lactone or derivative thereof and at least one pyrazino isoquinoline for the treatment of parasitic infestations in animals, wherein the composition is in the form of a topical solution.

### DETAILED DESCRIPTION OF THE INVENTION

In a first aspect the present invention refers to a pharmaceutical composition or formulation of an isoxazoline associated with a macrocyclic lactone or derivative thereof and a synthetic pyrazino-isoquinoline derivative for the treatment of parasitic infestations in minor animals, wherein the composition comprises the actives together with pharmaceutically and veterinarily acceptable excipients for topical administration.

The present invention refers to a composition or pharmaceutical formulation for topical application of an isoxazoline associated with a macrocyclic lactone or derivative thereof, and asynthetic pyrazino-isoquinoline derivative for the treatment of parasitic infestations in minor animals, wherein the composition comprises asisoxazoline or a salt or solvate of isoxazoline having as its structure the following formula:

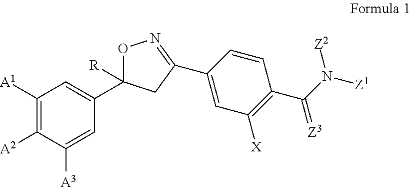

An isoxazoline, salt or solvate according to the structure of formula 1 wherein A1, A2 and A3 are selected from the group consisting of hydrogen, halogen and haloalkyl from 1 to 6 carbon atoms, e.g., halomethyl, halo ethyl, halopropyl; and wherein R is a haloalkyl from 1 to 6 carbon atoms, e.g., halomethyl; and wherein X is selected from the group consisting of hydrogen, halogen, alkyl from 1 to 6 carbon atoms, e.g., methyl, ethyl; haloalkyl from 1 to 6 carbon atoms, for example, halomethyl and haloethyl; and wherein Z1 and Z2 are substituents selected from the group consisting of hydrogen, alkyl from 1 to 6 carbon atoms, for example, but not limited to methyl, ethyl, propyl, butyl, tert-butyl, haloalkyl from 1 to 6 carbon atoms, for example, but not limited to halomethyl, haloethyl, halopropyl, halobutyl; 1- to 6-carbon alkyl-O-alkyl of 1 to 6 carbon atoms, e.g., methoxymethyl, ethoxyethyl; 1- to 6-carbon haloalkyl-O-alkyl of 1 to 6 carbon atoms, e.g., but not limited to halomethoxymethyl; ethoxymethyl, haloethoxymethyl, propoxymethyl, 1- to 6-carbon alkyl-aminocarbonyl 1- to 6-carbon alkyl, for example, but not limited to ethylaminocarbonylmethyl, ethylaminocarbonylethyl, dimethoxyethyl, propynylaminocarbonylmethyl; N-phenyl-N-methyl-amino, haloethylaminocarbonylmethyl, haloethylaminocarbonylmethyl, tetrahydrofuryl, methylaminocarbonylmethyl, (N,N-dimethylamino)-carbonylmethyl, propylaminocarbonylmethyl, cyclopropylaminocarbonylmethyl, propenylaminocarbonylmethyl, haloethylaminocarbonylcyclopropyl; and where Z3 consists of O and S and halogen is fluorine (F), chlorine (CI), bromine (Br) and iodine (I).

The present invention then comprises an isoxazoline, salt or solvate according to formula 1, wherein A1, A2 and A3 are halogen and may be fluoro, bromo or chlorine.

The present invention then comprises an isoxazoline, salt or solvate according to formula 1, wherein A1 and A2 are halomethyl and is trifluoromethyl.

The present invention then comprises an isoxazoline, salt or solvate according to formula 1, wherein R is monochloromethyl, trifluoromethyl, monochloro-difluoromethyl.

The present invention then comprises an isoxazoline, salt or solvate according to formula 1, wherein X is hydrogen, bromine, iodine, chlorine, methyl, ethyl, trifluoromethyl.

The present invention then comprises an isoxazoline, salt or solvate according to formula 1, wherein Z1 and Z2 are selected from the group consisting of hydrogen, methyl, haloethyl, halopropyl, halobutyl, methoxymethyl, halomethoxymethyl, ethoxymethyl, haloethoxymethyl, propoxymethyl, ethylaminocarbonylmethyl, ethylaminocarbonylethyl, dimethoxyethyl, propynylaminocarbonylmethyl, N-phenyl-N-methyl-amino, haloethylaminocarbonylmethyl, haloethylaminocarbonylethyl, tetrahydrofuryl, methylaminocarbonylmethyl, (N,N-dimethylamino)-carbonylmethyl, propylaminocarbonylmethyl, cyclopropylaminocarbonylmethyl, propenylaminocarbonylmethyl, haloethylaminocarbonylcyclopropyl; and wherein Z3 consists of oxygen (O) or sulfur(S).

The isoxazolines used in the present invention may have two or more conformational structures, but at least comprise a chiral carbon at position 5 of the isoxazoline ring. Other isoxazolines and their salts or solvates comprised in the present invention, are selected from fluralaner, sarolaner, afoxolaner and lotinaler, the like and/or combinations thereof.

The present invention refers to a composition or pharmaceutical formulation for topical application of an isoxazoline associated with a macrocyclic lactone or derivative thereof, and a pyrazino-isoquinoline derivative for the treatment of parasitic infestations in small animals, wherein the composition comprises a macrocyclic lactone selected from the group consisting of ivermectin, emamectin, eprinomectin, selamectin, doramectin, moxidectin, abamectin, the like and/or combinations thereof.

The present invention refers to a composition or pharmaceutical formulation for topical application of an isoxazoline associated with a macrocyclic lactone and a pyrazino-isoquinoline derivative for the treatment of parasitic infestations in small animals, wherein the composition comprises as a pyrazino-isoquinoline derivative praziquantel, or other antiparasitics such as pyrantel, febantel or combinations thereof. In the present invention at least one systemically active parasiticide can be used as an active agent against internal parasites, selected from the group consisting of one or more macrocyclic lactones, one or more benzimidazoles, levamisole, pyrantel, morantel, praziquantel, closantel, clorsulon, one or more amino acetonitrile active agents and one or more arylazole-2-yl cyanoethylamino active agents or combination thereof.

In this sense, the active ingredients may be present in a concentration between 0.1% and 50% based on the total weight of the composition. In one embodiment of the invention, the isoxazoline may be in a concentration between 2.5% and 40%. The pyrazino-isoquinoline-derived active ingredient, which may be for example, praziquantel, may be present in the composition in a concentration between 0.1% to 20%. The macrocyclic lactone is found in a concentration of 0.10% to 5%.

With regard to pharmaceutically acceptable excipients or components that may be in the pharmaceutical composition or formulation of the present invention, said excipients are selected from the group consisting of solvents, antioxidants, vehicle, flavorings, and mixtures thereof, among others. The term “pharmaceutically acceptable” is used to indicate that the component is suitable for use in a drug product, is compatible with the other components and is not harmful to the animal to which the product is administered.

The present invention also relates to a method for the prevention and/or treatment of infestation by external parasites, such as fleas, ticks, and mites, in domestic animals, in particular in dogs and cats, comprising the administration, preferably topical administration, more preferably administration by spot application, of a therapeutically effective amount of the liquid pharmaceutical composition as previously described.

According to this use, the preferred administration is the “spot-on” application, such that said medicament is intended to be applied by direct deposit on the skin of the animal, at the level of the shoulder blades or along a dorsal line starting from the base of the tail and going back up to the neck.

Thus, the present invention relates to a veterinary topical liquid in spot-on presentation whose pharmaceutical composition is comprised of a suitable organic solvent chosen from ethanol, isopropyl alcohol, butanol, isobutanol, isopropyl alcohol, propylene glycol, similar components, as well as of mixtures thereof, may be present in a concentration from 0.50% to 30.00%; preferably from 1.00% to 20.00%; more preferably from 2.00% to 10.00% wt/volume.

The use of the term “comprised by” should be interpreted in an inclusive and non-exclusive manner.

The composition of the present invention is also comprised of an organic vehicle chosen from propylene glycol monomethyl ether, dipropylene glycol n-butyl ether, ethylene glycol monomethyl ether, ethylene glycol monoethyl ether, diethylene glycol monoethyl ether and N-methyl 2-pyrrolidone, similar components, as well as mixtures thereof which may be present in a concentration of from 1.00% to 80.00%; preferably from 5.00% to 70.00%; more preferably from 10.00% to 60.00% wt/vol.

The antioxidants are advantageously chosen from ethyl or propyl gallate, alpha tocopherol, ascorbic acid, ascorbyl palmitate, monothioglycerol, butylated hydroxytoluene (BHT) and butylated hydroxyanisole (BHA), similar components; as well as from mixtures thereof and may be present in a concentration of 0.01% to 1.00%; more preferably from 0.02% to 0.5% wt/vol.

The composition may optionally contain a scent or aroma; to give fragrance and increase the comfort of the end user when applying the product to their pet and may be present in a concentration of from 0.01% to 15.00%; preferably from 0.05% to 10.00%; more preferably from 0.10% to 5.00% wt/vol.

In a second aspect, the present invention refers to a method of producing a pharmaceutical composition comprising an isoxazoline, a macrocyclic lactone and a pyrazino-isoquinoline derivative for the treatment of parasitic infestations in minor animals, wherein the method comprises the following steps:
a) Mixing the antioxidants with the selected vehicle(s), stirring until solution A is formed;
b) Adding the active ingredient Praziquantel to solution A, stirring until solution B is formed;
c) Adding the selected isoxazoline to solution B, stirring until solution C is formed;
d) On solution C incorporating the selected macrocyclic lactone, stirring until solution D is formed;
e) Mixing solution D with the selected aroma and the organic solvent, stirring until solution E is formed;
f) Leveling with the selected solvent; and
g) Checking the appearance, the solution obtained should be clear and free of particles.

A method for the prevention or treatment of external and internal parasitic infestations in minor animals wherein the method comprises administering a pharmaceutical composition or formulation of an isoxazoline associated with a macrocyclic lactone and a pyrazino-isoquinoline derivative for the treatment of parasitic infestations in small animals SDFDSD

In a fourth aspect, the invention further comprises the use of a topical composition of an isoxazoline associated with a macrocyclic lactone or derivative thereof and a synthetic pyrazino-isoquinoline derivative for the treatment of an animal in need of such treatment.

### Examples of Formulation or Compositions According to the Invention

Examples of the declared composition are shown below in Table No. 1. The water and alcohol solvents used in granulation evaporate during the process and are therefore not considered in the weight of the final composition.

TABLE NO 1
Examples of formulas with declared composition

| Component | A | B | C | D | E |
|---|---|---|---|---|---|
| Fluralaner | 28.125% | 28.125% | 28.125% | 28.125% | 28.125% |
| Moxidectin | 1.400% | 1.400% | 1.400% | 1.400% | 1.400% |
| Praziquantel | 9.000% | 9.000% | 9.000% | 9.000% | 9.000% |
| Butyl hydroxy | 0.050% | 0.050% | 0.100% | 0.100% | 0.100% |
| toluene | | | | | |
| Butyl hydroxy | 0.050% | 0.050% | 0.000% | 0.000% | 0.000% |
| anisole | | | | | |
| Isopropylalcohol | 5.000% | 2.500% | 0.000% | 0.000% | 0.000% |
| Ethyl alcohol | 0.000% | 0.000% | 4.000% | 2.500% | 5.000% |
| Propylene glycol | 5.000% | 2.500% | 0.000% | 2.500% | 5.000% |
| Diethylene glycol | 25.000% | 10.000% | 22.000% | 35.000% | 47.375% |
| N-methyl 2- | 22.375% | 42.375% | 31.375% | 17.375% | 0.000% |
| pyrrolidone | | | | | |
| Fragrance | 4.000% | 4.000% | 4.000% | 4.000% | 4.000% |
| Total: | 100.000% | 100.000% | 100.000% | 100.000% | 100.000% |

### Efficacy Studies of the Composition of the Present Invention

### Study Design

Each study animal received a pipette with the combination proposed by the present invention. The pipettes were applied to the skin at the base of the nape of the neck in its entirety. The treatment day was set as experimental day “0”.

A flea count was performed between experimental days “−7” and “0”, on the basis of which the animals were divided into two strata according to their parasite load, considering the median obtained as the division point.

After treatment, the animals were clinically evaluated within 15, 30, 60 and 120 minutes post-treatment to determine the possible presence of adverse effects.

The area counting technique was used to determine the flea load. Area counts were performed at six locations on each animal: neck, dorsal midline, tail base, left lateral, right lateral, and inguinal area as shown in Figure No. 1.

Evaluation of effectiveness was performed at 2 hours, 1, 2, 7, 14, 28, 28, 42, 56, 70 and 84 days post-treatment, based on the total number of fleas on each animal.

### Results

Summary of flea counts and effectiveness found for each group can be seen in Table N°2. The use of the composition of the present invention represents an effective alternative for the treatment and control of fleas in small animals for a minimum period of 12 weeks.

TABLE No 2
Effectiveness test results

| | | 2 | Days | | | | | | | | |
|---|---|---|---|---|---|---|---|---|---|---|---|
| | Basal | hours | 1 | 2 | 7 | 14 | 28 | 42 | 56 | 70 | 84 |
| Average count | 8.75 | 0.31 | 0.06 | 0 | 0 | 0 | 0 | 0 | 0 | 0 | 0 |
| Effectiveness | | 96% | 99% | 100% | 100% | 100% | 100% | 100% | 100% | 100% | 100% |

We claim:

1. A pharmaceutical composition, comprising as active ingredients an association of an isoxazoline with amacrocyclic lactone and a synthetic pyrazino-isoquinolinederivative.

2. The pharmaceutical composition according to claim 1, wherein the isoxazoline compound is a compound of formula 1:

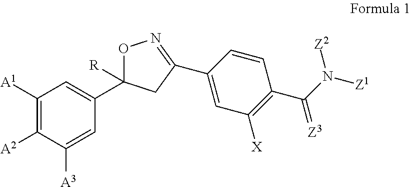

3. The composition according to claim 2, wherein A1, A2 and A3 are selected from the group consisting of hydrogen, halogen and halomethyl; and wherein R is a halomethyl; and wherein X is selected from the group consisting of hydrogen, halogen, methyl, halomethyl, ethyl and haloethyl; and wherein Z1 and Z2 are substituents selected from the group consisting of hydrogen, methyl, haloethyl, halopropyl, halobutyl, methoxymethyl, halomethoxymethyl, ethoxymethyl, haloethoxymethyl, propoxymethyl, ethylaminocarbonylmethyl, ethylaminocarbonylethyl, dimethoxyethyl, propynylaminocarbonylmethyl, N-phenyl-N-methyl-amino, haloethylaminocarbonylmethyl, haloethylaminocarbonylethyl, tetrahydrofuryl, methylaminocarbonylmethyl, (N,N-dimethylamino)-carbonylmethyl, propylaminocarbonylmethyl, cyclopropylaminocarbonylmethyl, propenylaminocarbonylmethyl, haloethylaminocarbonylcyclopropyl; and wherein Z3 consists of O and S.

4. The composition according to claim 2, wherein A1, A2 and A3 is halogen and may be fluorine, bromine, or chlorine.

5. The composition according to claim 2, wherein A1 and A2 is a halomethyl and is trifluoromethyl.

6. The composition according to claim 2, wherein R is monochloromethyl, trifluoromethyl, monochloro-difluoromethyl.

7. The composition according to claim 2, wherein X is hydrogen, bromine, iodine, chlorine, methyl, ethyl, trifluoromethyl.

8. The composition according to claim 2, wherein Z1 and Z2 are selected from the group consisting of hydrogen, methyl, haloethyl, halopropyl, halobutyl, methoxymethyl, halomethoxymethyl, ethoxymethyl, haloethoxymethyl, propoxymethyl, ethylaminocarbonylmethyl, ethylaminocarbonylethyl, dimethoxyethyl, propynylaminocarbonylmethyl, N-phenyl-N-methyl-amino, haloethylaminocarbonylmethyl, haloethylaminocarbonylethyl, tetrahydrofuryl, methylaminocarbonylmethyl, (N,N-dimethylamino)-carbonylmethyl, propylaminocarbonylmethyl, cyclopropylaminocarbonylmethyl, propenylaminocarbonylmethyl, haloethylaminocarbonylcyclopropyl; and wherein Z3 consists of oxygen or sulfur.

9. The composition according to claim 1, wherein the isoxazoline compound its salts or solvates are selected from fluralaner, sarolaner, afoxolaner, lotinaler, similar compounds or combinations thereof.

10. The composition according to claim 1, wherein the macrocyclic lactone compound is selected from ivermectin, emamectin, eprinomectin, selamectin, doramectin, moxidectin, abamectin, similar compounds or combinations thereof.

11. The composition according to claim 1, wherein the pyrazino-isoquinoline compound is praziquantel or another antiparasitic such as pyrantel, febantel or combinations thereof.

12. The composition according to claim 1, wherein the active ingredients may be present in a concentration between 1.00 and 50.00% based on the total weight of the composition; preferably from 2.5% to 40.00%.

13. The composition according to claim 1, wherein further comprising a vehicle selected from propylene glycol monomethyl ether, dipropylene glycol n-butyl ether, ethylene glycol monomethyl ether, ethylene glycol monoethyl ether, diethylene glycol monoethyl ether and N-methyl 2-pyrrolidone, similar components or mixtures thereof may be present in a concentration of 1.00% to 80.00%; preferably from 5.00% to 70.00%; more preferably from 10.00% to 60.00% wt/vol.

14. The composition according to claim 1, comprising a suitable organic solvent chosen from ethanol, isopropyl alcohol, butanol, isobutanol, isopropyl alcohol, propylene glycol, as well as mixtures thereof may be present in a concentration from 0.50% to 30.00%; preferably from 1.00% to 20.00%; more preferably from 2.00% to 10.00% wt/vol.

15. The composition according to claim 1, comprising an antioxidants which may be selected from ethyl or propyl gallate, alpha tocopherol, ascorbic acid, ascorbyl palmitate, monothioglycerol, butylated hydroxytoluene (BHT) and butylated hydroxyanisole (BHA); as well as mixtures thereof and may be present in a concentration of 0.01% to 1.00%; more preferably 0.02% to 0.5% wt/vol.

16. The composition according to claim 1, comprising a scent or aroma to provide fragrance and increase the comfort of the end user when applying the product to their pet and may be present in a concentration of from 0.01% to 15.00%; preferably from 0.05% to 10.00%; more preferably from 0.10% to 5.00% wt/volume.

17. A method of preparing a composition according to claim 1, comprising the following steps:
a) Mixing the antioxidants with the selected vehicle(s), stirring until solution A is formed;
b) Adding the active ingredient Praziquantel to solution A and stirring until solution B is formed;
c) Adding the selected isoxazoline to solution B, stirring until solution C is formed;
d) On solution C incorporating the selected macrocyclic lactone, stirring until solution D is formed;
e) Mixing solution D with the selected flavor and the organic solvent, stirring until solution E is formed; and
f) Leveling with the selected solvent.

18. A method for the prevention or treatment of external and internal parasitic infestations in minor animals wherein the method comprises administering a pharmaceutical composition or formulation of an isoxazoline associated with a macrocyclic lactone and a pyrazino-isoquinoline derivative for the treatment of parasitic infestations in minor animals.

19. The method according to claim 1, wherein the administration is topical and may preferably be spot-on application.
